(19) United States
(12) Patent Application Publication
(10) Pub. No.: US 2005/0181354 A1
(43) Pub. Date: Aug. 18, 2005

(54) Methods of assaying physiological states

(76) Inventor: Preston W. Estep III, Weston, MA (US)

Correspondence Address: Preston W. Estep III, 11 Willard Rd., Weston, MA, 02493, US

(21) Appl. No.: 10/780,232

(22) Filed: Feb. 18, 2004

(51) Int. Cl. C12Q 1/00; G06F 19/00; G01N 33/48; G01N 33/50

(52) U.S. Cl. 435/4; 702/19

(57) ABSTRACT

The present application provides, among other things, methods for monitoring biological states in a subject, as well as methods for monitoring the effect of interventions or therapies upon the biological states of a subject.

### CROSS-REFERENCE TO RELATED APPLICATIONS

This application is entitled to the benefit of U.S. Provisional Patent Application Ser. No. 60/447,677, Methods of Assaying Physiological States (Atty Docket No. LNT-P60), which was filed on Feb. 14, 2003.

### FEDERALLY SPONSERED RESEARCH

Not Applicable

### SEQUENCE LISTING OR PROGRAM

Not Applicable

### BACKGROUND OF THE INVENTION

1. Field of the Invention

This invention relates to methods used for assaying physiological states in humans, animals, and other organisms, specifically to determine certain important features of the physiological state related to health, fitness, disease, aging, and the like.

2. Background

The cells of living organisms produce a large diversity of RNA, protein, and other molecular species, in order to grow, reproduce, and respond to environmental cues. The production of RNA transcripts from genes in the living cell is known as transcription. The regulation of transcription is a complex process that allows the cell to grow, differentiate, and adapt to its environment. Transcript degradation is another level of regulation that allows the cell to respond to environmental cues. In most cell types these regulatory processes regulate the levels of transcripts in response to the needs of the cell. Therefore, analysis of a large number of transcripts from a cell, or population of cells from a specific tissue or grown in a defined condition, reveals important biological information about these cells, and about the organism from which the cells are derived.

In recent years many technologies have emerged for measuring the transcript levels of a large number of RNA transcripts in biological cells. Serial Analysis of Gene Expression and related technologies have allowed the analysis of RNA from genomes for which there is little or incomplete genomic sequence data, and a variety of microarrays (DNA “chips” or “arrays”) have emerged for measuring the abundances of RNA transcripts from mostly or completely sequenced genomes.

Microarray technology is useful for a variety of applications, including the identification of genes that are regulated by stresses and perturbations applied to cells, and the identification and analysis of genes in signaling pathways. The expression data derived from microarray analysis has facilitated the correlation of changes in gene expression within cells derived from a patient with disease states and prognoses. For example, the stratification of different types of cancers by transcript profiling allows more accurate prognoses and choice of treatments. The measurement of transcripts is not unique in this respect but it is the first method to have such predictive power. The measurement of other molecules should allow similar predictive power; however, these methods are less-well developed. Transcriptional responses to a large number of growth conditions have been used to group or cluster the more similar conditions or perturbations based on the similarities of the “transcript profile”, which refers to the analysis of many transcripts in an experiment.

In general transcript profiles from multicellular organisms are generated from cells that were taken from the organism itself, and the only reliable methods described thus far for diagnosing or stratifying disease are based upon the generation of expression profiles from cells taken directly from the affected tissue. For example, the current classification of tumorigenic or metastatic potential of cancers, or the stratification of cancers into therapeutic classes, requires that a transcript profile be generated from cells derived from the patient's tumor and then compared to transcript profiles of normal tissue, and to transcript profiles of other patients' cancers.

Similarly, in the transcript analysis of unicellular organisms, the RNA constituents of cells have been measured directly and used to infer something about the biology of the cells themselves. Other methods for generating profiles have focused on protein levels (instead of transcript levels), metabolite levels, protein modifications, or other cellular characteristics. The direct measurement of these molecules and their modifications should provide a great deal of insight into the physiological state of the organism; however, quantitative measurement of a large number of different species of these molecules is difficult. In each case, the focus has been on directly profiling cells from the subject organism, or directly profiling anonymous protein or blood chemistry markers associated with a given disease, or by measuring individual markers generated directly by the diseased or damaged cells or tissues.

The success of these types of profiling systems is limited and they suffer many shortcomings, including difficulty in comparing results across genetically diverse subjects, limitation in the sensitivity of detection of subtle changes, and in the case of human subjects, a general inability to obtain samples of, or transcript profiles from, non-diseased deep cells or tissues such as intestinal epithelial cells or hepatocytes. It is particularly unacceptable to sample from humans deep cells or tissues that are essentially irreplaceable or not easily regenerated such as cells of cardiac muscle or neurons from the spinal cord or brain. However, since the molecular profiles, including transcript profiles, in each of these cell types is unique, molecular profiles of these cell types would help to more clearly define a particular biological state and would allow the diagnoses of multiple diseases and non-disease biological and physiological conditions.

Improved methods for monitoring and defining physiological states of a subject, particularly a human subject, or animal model subject, may advance the ability of clinicians and researchers to measure the entire range of human physiological states, including the range of physiological states associated with health or disease status or biological age. Such methods would also allow the precise measurement of the physiological effects of any prescribed regimen on the overall health of the patient. This may be particularly important when a patient is taking a new or experimental drug. It would be useful, for example, if during treatment with the drug the health of the patient could be monitored and toxic effects upon the patient detected early in the treatment regimen. Such methods might allow very low levels of a therapy to be administered and responses to these low dosages could be extrapolated to predict reactions and outcomes. In addition, profiling systems employing more defined subject materials may permit more extensive and rigorous use of profiling methods in basic and clinical research.

Discussion or citation of a reference herein shall not be construed as an admission that such reference is prior art to the present application.

### SUMMARY

In certain aspects, the present application provides methods for monitoring and comparing physiological or biological states of a subject, including but not limited to, aging, hormonal status, infections or disease states and their progressions, or diet. In certain embodiments, the methods of the application involve obtaining one or more biological samples from a subject, using one or more of these biological samples to treat one or more cells of one or more types, measuring RNA, or protein, or metabolite abundances or activities in the cell, or of the medium in which it is grown, subsequent to the treatment of the cell to generate a “responsive cellular profile”. Optionally cells are grown in vitro. Optionally, responsive cellular profiles corresponding to one or more subjects and one or more biological states may be compared to generate “inferential molecular profiles” and “intensity correlation profiles”, each of which consists of multiple inferential molecular profiles such as that described above, which are obtained by measuring RNA, or protein, or metabolite abundances or activities in a cell, or of the medium in which it is grown, subsequent to the treatment of the cell with one or more biological samples obtained either from another subject, or subjects, or from said subject at multiple other times or physiological states, including but not limited to, different age states, during the course of an infection or disease, or subsequent to some notable physiological change in the subject. Certain embodiments of the present application also provide methods for generating molecular profiles from the patient's or subject's cells in a similar fashion, as both a calibration and as an adjunct method to the primary method, to aid in diagnosing certain biological states, and for determining therapy types and levels.

In certain aspects, the present application provides methods for monitoring the efficacy or response to a perturbation, therapy, intervention, or treatment upon a subject, in order to alter the physiological state, such as those described above. The methods of the application involve obtaining an inferential molecular profile, by measuring RNA, or protein, or metabolite abundances or activities in a cell, or of the medium in which it is grown, subsequent to the treatment of the cell with one or more biological samples obtained from the subject, and comparing said inferential molecular profile to one or more intensity correlation profiles, each of which consists of multiple inferential molecular profiles such as that described above, which are obtained by measuring RNA, or protein, or metabolite abundances or activities in a cell, or of the medium in which it is grown, subsequent to the treatment of the cell with one or more biological samples obtained either from another subject, or subjects, or from said subject at multiple times, intensities, or doses of a perturbation, therapy, intervention, or treatment. The present application also provides methods for generating molecular profiles from the patient's or subject's cells in a similar fashion, as both a calibration and as an adjunct method to the primary method, to aid in diagnosing certain biological states, and for determining therapy types and levels.

Certain methods of the application are based at least in part on our need to measure human aging, the development of pre-disease states and diseases of aging, hormonal imbalances that occur with age, and interventions in the aging process, and to compare these methods with similar treatments successfully applied to model organisms, such as dietary interventions, e.g. calorie restriction. Certain methods are also based in part on the discovery that changes to physiology that accompany changes in diet, or drug treatment, or course of a disease induce changes to various constituents of the cells of the organism, such as changes of protein function or abundance, and that these changes result in characteristic changes in the transcriptional activity of genes other than that encoding the changed protein, and that such changes can be used to define a “signature” transcript profile that is correlated with the physiological state, dietary status, or progression of a particular disease state or therapy. This is true even if there is no change or disruption in the function or abundance of proteins associated with the disease state. Thus, various methods of the present application are different from and independent of monitoring protein function.

In certain methods of this application, cells are treated in vitro with biological samples obtained from the subject, and the cells themselves act as sensitive detectors of physiological change in the subject. The biological samples may also be of many different types: urine, mucous, tears, blood, saliva, feces, peritoneal fluid, cerebrospinal fluid, amniotic fluid, etc. In further embodiments, molecular profiles obtained from the subject's cells are used in the present application to measure a variety of parameters of the physiological state of the subject not directly necessarily attributable to a disease or to the treatment of a disease, and in particular, may be irrelevant to the health or disease state of the cells themselves.

In additional embodiments, methods of the application can be used to monitor several separable physiologic states, diseases and/or therapies simultaneously, such as biological age, dietary status, hormonal status, progression of a disease, and/or efficacy of a therapy used to treat the disease.

Certain detailed methods of the application provide, first, methods for determining or monitoring the level of one or more physiological states, including but not limited to, normal “baseline” states, stages of biological age and aging, states caused by infection or disease, physiological states induced by toxic exposures, or diet, upon a subject by: (i) obtaining from a subject one or more biological samples, including but not limited to blood, urine, feces, or skin secretions; (ii) treating cells of one or more types with one or more of these biological samples or their fractions or extracts thereof; (iii) measuring abundances of, or alterations to, cellular constituents of said cells subsequent to said treatment such that an inferential molecular profile of the physiological state of the subject is obtained; (iv) obtaining interpolated intensity correlation profiles for each physiological state being analyzed by, first, obtaining inferential molecular profiles from an analogous subject at a plurality of different ages or times, including but not limited to various times during the course of an infection or disease, or at a plurality of levels of each physiological state, and second, interpolating the thereby obtained inferential molecular profiles; and (v) determining the interpolated intensity correlation profile for each physiological state for which similarity is greatest between the inferential molecular profile and a combination of the determined interpolated intensity correlation profiles, according to some objective measure. The intensity or level of a particular physiological state is thereby indicated by the phenotypic intensity correlated to the thus determined interpolated intensity correlation profile for that physiological state. Embodiments of the application further provide methods for obtaining molecular profiles from the patient's cells which yield, in a manner similar to that described above, information that is useful as both a calibration, and as an adjunct method to the primary methods of the application described above, wherein this adjunct method yields additional measurements of important parameters of certain biological states.

Certain aspects of the present application also provide methods for determining or monitoring the effect of, or response to, a therapy or treatment upon a subject by: (i) obtaining one or more biological samples, including but not limited to blood, urine, feces, or skin secretions, from a subject undergoing one or more therapies or treatments, including but not limited to those involving drugs, changes in or supplementation to diet, or application of topical therapies or formulations, personal care or skin creams; (ii) treating cells of one or more types with one or more of these biological samples or their fractions or extracts thereof; (iii) measuring abundances of, or alterations to, cellular constituents of said cells subsequent to said treatment such that an inferential molecular profile of the physiological state of the subject is obtained; (iv) obtaining interpolated intensity correlation profiles for each therapy or treatment by, first, obtaining inferential molecular profiles from an analogous subject or subjects at a plurality of different intensities, and/or dosages, and/or times, before, and/or during and/or after said therapy, intervention, perturbation, or treatment, and second, interpolating the thereby obtained inferential molecular profiles; (v) determining the interpolated intensity correlation profile for each therapy or treatment for which similarity is greatest between the inferential molecular profile and a combination of the determined intensity correlation profiles, according to some objective measure. The effect of a particular therapy is thereby indicated by the level of effect correlated to the thus determined interpolated intensity correlation profile for that therapy. In various aspects of this second embodiment, the methods of the application can be used to monitor beneficial effects or adverse effects of therapies. For example, the methods can be used to monitor toxic effects of a therapy. Embodiments of the application further provide methods for obtaining molecular profiles from the patient's cells which yield, in a manner similar to that described above, information that is useful as both a calibration, and as an adjunct method to the primary methods of the application described above, wherein this adjunct method yields additional measurements of important parameters of certain biological states.

In various aspects of the above embodiments, the inferential molecular profile can be determined by measuring changes within the cells and treated with one or more biological samples from the subject, and these changes include but are not limited to, gene expression, protein abundances, protein activities, protein modifications, metabolite abundances, or a combination of such measurements. In a preferred aspect of the above embodiments, the determined interpolated response profile for each physiological state or perturbation or treatment or therapy is the interpolated intensity correlation profile which minimizes an objective function of the difference between the inferential molecular profile and a combination of the determined interpolated intensity correlation profiles for all physiological states or perturbations or treatments or therapies being evaluated. Molecular profiles from the patient's cells provide additional diagnostic information, information that is useful as both a calibration, and as an adjunct method to the primary methods of the application described above, wherein this adjunct method yields additional measurements of important parameters of certain biological states.

### DRAWINGS

Not Applicable

### DETAILED DESCRIPTION

1. Definitions

For convenience, certain terms employed in the specification, examples, and appended claims are collected here. Unless defined otherwise, all technical and scientific terms used herein have the same meaning as commonly understood by one of ordinary skill in the art to which this application belongs.

A “cell population” is more than one cell. A heterogeneous cell population is a cell population comprising more than one cell type. A homogeneous cell population is a cell population that comprises, as far as practicable, a single cell type. The term “cell type” includes genetically similar cells, e.g., from a cultured cell line.

As used herein, a “biological sample” or “sample” is one or more samples of biological material obtained by from a subject, or their secretions, extracts, and fractions thereof. These include, but are not limited to, urine, mucous, tears, blood, lymphatic fluid, saliva, phlegm, sweat, skin oil and other secretions, feces, vomitus, milk, semen, vaginal secretions, peritoneal fluid, cerebrospinal fluid, sebum, amniotic fluid, blister fluid, pus, pleural fluid, synovial fluid, tissue and cell extracts, and other bodily fluids. The subject may be an organism, an isolated organ, tissue, or cells, including cells cultured in vitro, and the biological samples obtained from these subjects include, but are not limited to, bodily fluids and, or secretions from the organ, cells or tissue. As used herein the “subject's cells” are cells that are part of the subject, or are derived from the subject. These include but are not limited to cells from blood, epithelium, lymph or lymphatic system, skin, adipose, brain, liver, skeletal muscle, kidney, breast, and lung. As used herein, “bioactive agent” is any agent or physical parameter, including but not limited to drugs, organic and inorganic compounds, synthetic or natural compounds, biomolecules such as protein, DNA, or RNA, physical parameters such as radiation, heat, or cold, or other agents that causes a measurable biological response in the assay cells.

A “biological state” is essentially any characteristic of an organism, and the biological state may be completely unknown, though typically at least rudimentary information about the biological state will be available, such as species, age and sex (where relevant). The biological state may also be very well characterized. The description of the biological state may change over time as more is learned about the state of the subject before, during, or after the time the sample is taken. For example, a frozen blood sample from a subject may later be assessed for markers of myocardial infarction (e.g. troponin levels), thus allowing a classification of the biological state of the subject as likely or unlikely myocardial infarction. A biological state may also be a physiological state. A “physiological state” refers to any measurable state of the subject's physiology. These states include nutritional status, hormonal status, biological age and rate of aging, disease, illness, infection, general cardiovascular or pulmonary fitness due to exercise and/or biological age, etc. It is expected that physiological state is somewhat dynamic and that changes in physiological state may be effected by multiple factors including but not limited to diet, exercise, genetic modification, sexual activity, sleep or rest, topical and/or parenteral and/or oral therapies or drugs, infection, or the development of a disease or illness. The overall physiological state is expected to be composed of subclasses of physiological states of the various cells, tissues, and organ systems of the subject. The present application is useful for the determination of the health state of these subclasses and their responsiveness to various interventions and therapies, as each type of cell, tissue, or organ will exert unique effects upon the composition of the biological sample used for treatment, and these unique effects will be to some degree measurable by their effect on the cellular state of cells. A physiological state may change rapidly in a subject over time. This physiological state is measurable, in at least some aspects, by the effect of a biological sample derived from the subject upon the cellular state of a cell.

A “cellular profile” is a set of measurements (optionally quantitative measurements) and/or observations of a plurality of cellular constituents. A profile may comprise as few as one and as many as 5, 10, 20, 50, 100, 500, 1000, 5000, 10000 or more constituents. Cellular constituents may include RNA, or protein abundances; RNA or protein activity levels; RNA, DNA, or protein modification states (e.g., methylation, phosphorylation, or glycosylation). Such constituents may also include small molecule abundance, activity, and modification state. The measurements and/or observations made on the state of these constituents can be of their abundances (i.e., amounts or concentrations in a cell), or their activities, or their states of modification (e.g., phosphorylation), or other measurement relevant to the characterization of the response of the cell to treatment with a drug or nutrient or biological sample. A “responsive cellular profile” is a cellular profile of cells after exposing the cells to a sample obtained from a subject. A responsive cellular profile “corresponds” to the biological state of the subject from which the sample was obtained. A “control cellular profile” is a profile obtained from cells that were not exposed to the sample.

A “cellular state” means the state of a collection of cellular constituents, which are sufficient to characterize the cell for an intended purpose, such as for characterizing the effects of a biological sample or variation of nutrient composition or a drug.

The term “including” is used herein to mean, and is used interchangeably with, the phrase “including but not limited to”.

A “predictive profile” is a profile that is predicted to correspond to a particular biological state. Predictive profiles may be calculated from an inferential set or from direct interpolation or extrapolation from a plurality of responsive cellular profiles.

A “cellular profile” or “profile” is a plurality of cellular constituents and associated measurements, observations or predicted, inferred or calculated values.

A “set of cellular constituents” or “set” includes the identity of one or more cellular constituents that are useful for a particular purpose. An “inferential set” is the identity of one or more cellular constituents the measurement or observation of which is informative of a biological state. An “inferential set” may also include a quantitative or qualitative description of the relationship between the cellular constituents and a range of biological states.

A “therapy” or “therapeutic regimen”, as used herein, refers to a regimen of treatment intended to reduce or eliminate the symptoms associated with less preferable physiological states, such as biological age, aging, toxification, or disease. A therapeutic regimen will may comprise dietary changes, ingestion of dietary supplements, application of topical compounds, genetic therapy, or a prescribed dosage of one or more drugs, prehormones, or hormones, among others.

2. Overview

In one aspect, the present application includes methods for generating a cellular profile that corresponds to a biological state of a subject. In further aspects, the application relates to methods for using cellular profiles to monitor a biological state, including, for example, a physiological state and the efficacy of one or more therapies upon a subject. In yet additional aspects, inventive methods include comparing cellular profiles that correspond to biological states in order to, for example, deduce information about the molecular nature of a biological state or infer some aspect of a biological state in a subject.

In certain embodiments, the methods involve the use of a sample obtained from the subject, for the purpose of treating cells with the sample, and measuring a plurality of cellular constituents to obtain a responsive cellular profile. The responsive cellular profile provides information regarding the biological state of the subject at the time the sample was obtained. In this manner, the responsive cellular profile is said to “correspond” to a biological state of the subject from which the sample was obtained. Optionally, a responsive cellular profile is compared to a “control cellular profile”, meaning a cellular profile obtained from cells that were not subjected to treatment with a sample. Refined profiles resulting from such comparisons are included in the term “responsive cellular profile”. Optionally, the biological state of the subject may be inferred by comparing the corresponding responsive cellular profile to one or more other responsive cellular profiles obtained in a similar manner, from either the same subject or from one or more other subjects, and which are associated with various degrees, or intensities, or stages, of characterized biological states, such as biological age, rate of aging, disease, course of infection, nutritional status, etc., or which are associated with controllable or inducible physiological states, such as the ingestion of known foods or food components, the administration of known levels of topical or oral therapies or treatments for known or suspected diseases, disorders, or ailments, exercise, etc.

Certain methods of the application relate to the creation of two or more related sets of information: (1) information about effects of a sample on a cell population and (2) information about the subject from which the sample was obtained. Each set of information may be analyzed to provide further information. For example, the comparison of clinical information about disease progression to corresponding responsive cellular profiles may provide information about the molecular mechanisms (e.g. genes or proteins involved) by which disease progression occurs. As another example, the analysis of responsive cellular profiles corresponding to a subject of undetermined biological state (e.g. stage of disease) may allow assignment of an inferred biological state. In further embodiments, the application relates to the creation of sets of responsive cellular profiles, each corresponding to a sample from a subject. The biological state of the subject may be known, partly described or unknown and any known clinical information about the subject generally (including past history and eventual outcome) or specifically at the time the sample was taken may be linked to the responsive cellular profiles. The sets of profiles and corresponding clinical information may be compared to deduce statistically robust relationships between one or more aspects of cellular response and biological states. Optionally, the various sets of information may be organized into a relational database.

In certain aspects, methods of the application employ a body fluid or other easily-obtained samples from a subject to assay or infer the subject's physiological state, including, for example, the state of some subset of the subject's cells. Responsive cellular profiles may be generated using such samples from subjects receiving one or more of the following exemplary therapies: a defined diet, dietary change, dietary supplements, drugs and their metabolites, hormones, pre-hormones, bioactive peptides, other bioactive agents, toxins, herbal and nutritional supplements, or other ingested or topical treatments, and the profiles may be monitored and compared to, and grouped or classified with, profiles that correspond to other therapies. A collection of such responsive cellular profiles may be used to predict likely short and/or long-term effects of treatments with currently unknown effects. The measurement of these effects, and their comparison to the effects of known drugs, therapies, diets, toxins, or other treatments, may also be used in determining accurate dosing regimens for drugs and other therapies, more accurate prognoses for these treatments and other physiological perturbations.

The responsive cellular profile resulting from the treatment of cells with drugs, hormones, vitamins, radiation, or other treatments, either alone, or together with a biological sample from the subject, will aid in the identification of which treatments result in a desired responsive cellular profile. For example, if a subject is deficient in growth hormone, then treatment of cells with a biological sample from the subject will result in a responsive cellular profile, and the comparison of this profile to a responsive cellular profile obtained from cells treated with growth hormone, or with growth hormone together with a second biological sample from the subject, will show the subject to be deficient in growth hormone, especially when compared to the response profiles of other subjects who display a range of levels of growth hormone. The responsive cellular profiles of known mutations, drugs, hormones, and other treatments serve to identify the biological pathways affected by these agents, and they allow the identification of biological processes, agents, cell types, and drug and therapeutic targets.

In certain embodiments, methods of the application may be used to determine or otherwise obtain information about, a biological state of a subject, such as a physiological state. Information about the biological state of a subject may be determined by detecting changes in a sample from the subject that tend to be coincident with a biological state. Changes in the sample may be detected by generating a responsive cellular profile using a sample from the subject. It is considered here that disease states of various kinds, many stages of biological age, the intrinsic rate of aging, infection, resistance to disease and infectious agents of various kinds, nutritional status, among others, are differentiable physiological states, as is the degree of subject's physiological response to one or more therapies. Thus, the present application also provides methods for determining or monitoring efficacy of a therapy or therapies (i.e., determining a level of therapeutic effect) upon a subject. In a specific embodiment, the methods of the application can be used to assess therapeutic efficacy in a clinical trial, e.g., as an early surrogate marker for success or failure in such a clinical trial.

In certain embodiments, information about the biological state of a subject may be obtained by comparing the responsive cellular profile to one or more other responsive cellular profiles corresponding to one or more biological states. Preferably, similar aspects of cellular states are compared, e.g., if the first responsive profile is a transcriptional profile, it is preferable to compare this to other responsive cellular profiles that comprise transcriptional profiles. The additional cellular profiles for comparison may be obtained from the same subject and/or other subjects, and these subjects may be selected randomly, or optionally the subjects may be selected to have certain characteristics in common with the first subject and/or certain characteristics that are different from the first subject. For example, if a subject is to be monitored for effectiveness of therapy, the first responsive cellular profile may be compared against responsive cellular profiles corresponding to subjects who experienced a range of effects from the same or a similar therapy. Similarly, if disease progression is to be assessed, the first responsive cellular profile may be compared against profiles corresponding to subjects having a range of progression stages of the same disease or a similar disease. Where one or more profiles are compared, they may be used to generate a new representation termed the “similarity index” which is a representation of the similarity between the profiles. Comparisons may be done by correlative methods, clustering methods, or other methods known in the art. A biological state may be inferred by simply identifying the most similar responsive cellular profile and inferring that the corresponding biological state is also the most similar. Optionally, a plurality of responsive cellular profiles corresponding to known biological states are used to define the set of cellular constituents that are informative of biological state, and the quantitative relationship between the measurement o or observation of each informative cellular constituent and the biological state. A set of cellular constituents with these properties is termed an “inferential set”. A responsive cellular profile corresponding to an unknown biological state may then be analyzed using the inferential set. In other words, the measurements or observations of the informative cellular constituents from the profile of unknown biological state are compared to the inferential set in order to infer a predicted biological state. An inferential set may be interpolated or extrapolated to provide predictive profiles for biological states that are intermediate in degree between or greater or lesser than the biological states for which comparison indices have been gathered. In cases where therapeutic efficacy is to be monitored, a responsive cellular profile may be compared to responsive cellular profiles from subjects in which the therapy had a beneficial effect, an adverse effect, such as a toxic effect, or both beneficial and adverse effects.

In certain embodiments a plurality of responsive cellular profiles that correspond to a range of related biological states may be analyzed to create an “inferential set”. For example, a series of responsive cellular profiles corresponding to increasingly severe disease states may be analyzed to identify the levels of cellular constituents predictive of (e.g. correlated with) severity of disease state. A new responsive cellular profile may then be mapped onto the inferential set to determine the predicted severity of disease state. In another example, responsive cellular profiles are obtained that correspond to subjects having positive and/or negative effects from a therapeutic regimen. The profiles may be analyzed to identify the inferential set of cellular constituents that are most useful for classifying the effect of the therapeutic regimen. A new responsive cellular profile may be processed using the clustering (or classifying) inferential set and the predicted efficacy of the therapeutic regimen determined.

Comparative embodiments of the application may comprise monitoring a plurality of physiological states or therapies in an individual subject; for example in a subject monitored at a variety of biological ages, or having several genetic mutations that are each associated with a particular disease, or in a subject undergoing several therapeutic regimes simultaneously (for example, a patient taking several drugs, each of which has a different effect). Accordingly, responsive cellular profiles are obtained individually for a sufficient subset of disease states or a sufficient subset of states of response to one or more therapy, to allow interpolation or extrapolation resulting in predictive cellular profiles for the desired broader range of potentially observable pluralities of physiological states or therapies.

Similarly, in certain embodiments, cellular constituents in a responsive cellular profile may be compared to cellular constituents varying in other responsive cellular profiles of known biological state in order to find a level of a biological state or effect of a therapy, for which the responsive cellular profile matches all or substantially all of the predictive constituents of a corresponding cellular profile. If a plurality of physiological states or therapies is being monitored, then the responsive cellular profile is compared to some combination of the individual responsive cellular profiles for each physiological state or therapy. Substantially all of a responsive cellular profile is matched by another responsive cellular profile when most of the cellular constituents that vary with the biological state (i.e. have predictive value) are found to have substantially the same value in the two profiles. Cellular constituents have substantially the same value in the two profiles when differences between the normalized sets of data are statistically insignificant given experimental error.

In a preferred embodiment, comparison of a responsive cellular profile with a curve that relates biological state to one or more predictive cellular constituents is performed by a method in which an objective measure of difference between a measured responsive cellular profile and a predictive cellular profile determined for some known perturbation level, i.e., for some level of a particular physiological state or therapeutic efficacy, is minimized. The objective measure is minimized by extracting the predictive cellular profile from the curves that relate biological state to one or more predictive cellular components at the perturbation level at which the objective measure of distance is minimized. Minimization of the objective measure can be performed by standard techniques of numerical analysis. See, e.g., Press et al., 1996, Numerical Recipes in C, 2nd Ed. Cambridge Univ. Press, Ch. 10.

In certain embodiments, responsive cellular profiles are obtained from cells co-treated with a biological sample and a drug, toxin or other compound. The responsive cellular profiles obtained may be compared to the cellular profiles obtained from a subject treated with the same drug, toxin or other compound. “Pre-metabolized” drugs or other compounds may be used to treat the cells, with pre-metabolism accomplished, for example, by treating the compound with liver homogenate. Comparison of co-treatment with treatment of a person allows separation of primary and secondary effects of a drug. For example, glucose treatment of a person causes a particular response in an individual that includes hormonal changes, while glucose treatment of cells co-treated with serum from a patient low in glucose, with or without additional insulin, allows a separation of the direct effects of glucose and the metabolic influences of the hormones that accompany increased glucose in vivo.

In certain embodiments, a cellular response profile is obtained by contacting cells with samples from a plurality of subjects having defined ages. Cellular profiles of this type may be compared to deduce senescence-related factors present in the samples. In a preferred embodiment, the cellular profile is inspected for evidence of a factor secreted by a senescent cell that is present in a sample from a subject. Similarly, profiles of cells from a plurality of subjects having defined ages may be obtained and compared in order to, for example, identify transcripts that are expressed in an age-regulated manner. Subjects may also be selected to represent a likely range of senescence, ranging from non-senescent to pre-senescent to senescent. In certain embodiments, cellular profiles and responsive cellular profiles are inspected for evidence of cell damage, death, and apoptosis, and necrotic and apoptotic tendencies may be compared across various physiological states, including age, inflammatory disease conditions, etc. In certain embodiments, a cellular profile or cellular response profile is inspected for evidence of mitochondrial dysfunction. Mitochondria are vulnerable to oxidative damage and mitochondrial dysfunction is associated with cell senescence. Additional physiological states that may be particularly assessed include developing embryos or fetuses, that may be biopsied directly (non-human animals) or that may be monitored through, for example, the amniotic fluid. Inspection of cellular response profiles in cells contacted with an embryonic or fetal sample may reveal birth defects, fetal or embryonic distress, predictive information on time of birth, birth weight and health of the mother. Likewise, maternal samples may be contacted with cells and cellular profiles measured for the purpose of monitoring pregnancy.

In certain embodiments, cells to be contacted with a sample are a single homogeneous population of cells, such as a cultured cell line. In certain embodiments, cells to be contacted with a sample are heterogeneous. Optionally, cells to be contacted with a sample are a set of distinct cell types, preferably arranged in an array. For example, cells engineered to express a reporter gene from different promoters may be placed in an array and thereby provide simultaneous readout corresponding to activation of a variety of promoters in response to a sample. For example, cells engineered to have reporter genes driven by a variety of senescence and apoptosis-related promoters may be used. As another example, cells to be contacted may be cell lines or cultured cells from a variety of different tissues, providing a multi-cell type survey of the response to a sample.

3. Subjects and Samples

A subject may be any unicellular or multi-cellular organism, optionally an animal, particularly a mammal, and may also be one or more portions of such organisms, such as an isolated organ, tissue or cells. In certain embodiments, the subject is a human. With respect to the embodiment in which a non-human animal is used as the subject, animals of veterinary, farm, or domestic, importance, such as chickens, cows, pigs, dogs, cats, etc., and those commonly used as models for human physiological function and disease, such as primates, mouse, rat, and nematode, are all exemplary subjects. The subject need not be living at the time of collection of the biological sample. This is useful in a variety of situations, including but not limited to the forensic analysis of the subject. Methods disclosed herein may allow comparison between different subject species where appropriate.

In certain embodiments, methods disclosed herein employ samples (or fractions thereof) pooled from a single subject and/or pooled from a plurality of subjects. This makes the methods described herein particularly useful when dealing with individual cultured cells, but pooling of individuals to comprise a subject is useful in the analysis of any type of organism. In certain embodiments, samples from subjects sharing a particular biological state, such as a defined age range, are pooled. Pooling of subjects based on a particular variable is an effective technique for controlling for variables in a population of subjects. In an exemplary embodiment, samples are pooled from a group of subjects having a first age range, and other samples are pooled from a group of subjects having a second age range. The cellular profiles or responsive cellular profiles from each pool are compared to assess age-related changes, while controlling for other variables within the subject populations.

As used herein, a “biological sample” or “sample” is one or more samples of biological material obtained by from the subject, or their extracts and fractions thereof. These include, but are not limited to, urine, mucous, tears, blood, lymphatic fluid, saliva, phlegm, sweat, skin oil and other secretions, feces, vomitus, milk, semen, vaginal secretions, peritoneal fluid, cerebrospinal fluid, sebum, amniotic fluid, blister fluid, pus, pleural fluid, synovial fluid, tissue and cell extracts, and other bodily fluids. When organs, tissues and cells are available from an individual or subject, then the tissues or cells, or their fractions, extracts, secretions, and the like, are suitable biological samples.

In certain embodiments, bodily fluids and other biological samples derived from the subject may serve as an accessible surrogate for these cells or tissues that are not easily obtained from a subject. While not wishing to be bound to theory, it is expected that, since a subject's cells are bathed in body fluids, take up nutrients and metabolites from the fluids, and secrete into these fluids a variety of wastes, hormonal and other chemical messengers, growth factors and other proteins, and breakdown products of drugs and toxins, and the biological activity of the secreted products in such fluids will cause a biological response that is diagnostic of the state of the in vivo cells. The measurement of some of these individual secreted factors, such as serum proteins, are used to infer toxic effects of a given treatment upon some organ. For example, circulating serum concentrations of alpha-fetoprotein or alkaline phosphatase are commonly used to monitor liver damage (see, e.g., Izumi, R. et al., 1992, Journal of Surgical Oncology 49:151-155). The action of the widely-used immunosuppressants Cyclosporin A and mycophalote mofetil have also been monitored using assays for the activities of the target enzymes calcineurin and inosine monophosphate, respectively (see, Yatscoff, R. W. et al., 1996, Transplantation Proceedings 28:3013-3015). In another example, characterization of cerebrospinal fluid may be quite informative about neural cells in contact with the fluid.

In some methods of the present application, cells are exposed to the biological sample (optionally, as noted above, the sample employed is an extract or fraction of material obtained from the subject). For example, the sample may be added to the media in which cells are grown, mixed with media or with a variety of other ingredients before, during, or after initial exposure to the cells. This exposure of the assay cells to the sample is a “treatment”, and cells exposed in such a manner are said to be “treated” by the sample.

4. Cellular Profiles

In some aspects, the methods of the present application include methods of measuring and observing a plurality of cellular constituents to generate a cellular profile. Optionally a cellular profile may be used to assign a cellular state to the profiled cells. A cellular state (or state of a cell), as used herein, is taken to mean the state of a collection of cellular constituents, which are sufficient to characterize the cell for an intended purpose, such as for characterizing the effects of a biological sample or variation of nutrient composition or a drug. The measurements and/or observations made on the state of these constituents can be of their abundances (i.e., amounts or concentrations in a cell), or their activities, or their states of modification (e.g., phosphorylation), or other measurement relevant to the characterization of the response of the cell to treatment with a drug or nutrient or biological sample. In various embodiments, this application includes making such measurements and/or observations on different collections of cellular constituents. These different collections of cellular constituents are also called herein different types of cellular profiles that may reflect different aspects of the cellular state. The term “cellular profile” also includes any representation of measurements and/or observations of cellular constituents, including representations where a baseline subtraction or a comparison to a control cellular profile has been performed. The term “cellular profile” is not, therefore, limited to the raw data obtained from measurements and/or observations of a plurality of cellular constituents.

Although for simplicity this disclosure often makes references to single cell (e.g., “RNA is isolated from a cell”), it will be understood by those of skill in the art that more often any particular step of the application will be carried out using a plurality of genetically similar cells, e.g., from a cultured cell line. Such similar cells are called herein a “cell type”. Such cells are derived either from naturally single celled organisms, or derived from multi-cellular higher organisms (e.g., human cell lines).

A transcriptional profile may be generated and used to deduce the transcriptional state of the cell. The transcriptional state of a cell includes the identities and abundances of a plurality of RNA species, especially mRNAs, in the cell under a given set of conditions. Preferably, a substantial fraction of all constituent RNA species in the cell are measured, but at least, a sufficient fraction is measured to characterize the action of a biological sample or other test agent, such as a nutrient or drug of interest. A transcriptional profile may be conveniently generated by, e.g., measuring cDNA abundances by any of several existing gene expression technologies.

Another type of cellular profile usefully measured in the present application is a translational profile. The translational profile of a cell includes the identities and abundances of the constituent protein species in the cell under a given set of conditions. Preferably, a substantial fraction of all constituent protein species in the cell are measured, but at least, a sufficient fraction is measured to characterize the action of a biological sample or nutrient or drug of interest. As is known to those of skill in the art, the transcriptional state is often representative of the translational state.

Another type of cellular profile usefully measured in the present application is association of transcription factors and chromatin and chromatin-associated proteins with DNA. These associations are measurable in multiple ways known to those skilled in the art, such as, e.g., chromatin immunoprecipitation and DNA chip hybridization of the DNA fragments. Preferably, a substantial fraction of all the upstream and other regulatory regions of all predicted genes of the cell are measured, but at least, a sufficient fraction is measured to characterize the action of a biological sample or nutrient or drug of interest. As is known to those of skill in the art, the association of these protein factors with DNA is often representative of the transcriptional state.

Other types of cellular profiles, reflecting other aspects of cellular state may be employed in the methods of the application. For example, the activity state of a cell, as that term is used herein, includes the activities of the constituent protein species (and also optionally catalytically active nucleic acid species) in the cell under a given set of conditions. As is known to those of skill in the art, the translational state is often representative of the activity state. Other exemplary aspects of a cellular state include the phosphorylation state of cellular polypeptides, glycosylation state of cellular polypeptides, metabolite abundances, etc.

In certain embodiments, methods of the application are adaptable, where relevant, to “mixed” aspects of a cellular state in which measurements of different aspects of the cellular state of a cell are combined. For example, in one mixed aspect, a cellular profile may comprise the abundances of certain RNA species and of certain protein species combined with measurements of the activities of certain other protein species.

In certain embodiments, cellular profiles are determined from a population of cells, and the population of cells may be homogeneous or heterogeneous. In such cases the terms cellular profile and cellular state will be understood to refer to the profile and inferred state of the cell population, although a profile obtained from a heterogeneous cell population may be analyzed to deduce profiles and states that apply to each represented cell type individually.

Perturbations of a cell may affect many constituents of whatever aspects of the cellular state are being measured and/or observed in a particular embodiment of the present application. In particular, as a result of regulatory, homeostatic, and compensatory networks and systems known to be present in cells, even the direct disruption of only a single constituent in a cell, without directly affecting any other constituent, may have complicated and often unpredictable indirect effects.

The inhibition of a single, hypothetical protein, protein P is considered herein as an example. Although the activity of only protein P is directly disrupted, additional cellular constituents that are inhibited or stimulated by protein P, or which are elevated or diminished to compensate for the loss of protein P activity will also be affected. Still other cellular constituents will be affected by changes in the levels or activity of the second tier constituents, and so on. These changes in other cellular constituents can be used to define a “signature” of alterations of particular cellular constituents that are related to the disruption of a given cellular constituent. A responsive cellular profile obtained after a perturbation provides a record of the cellular state after a perturbation, and it is possible in many instances to deduce information about the perturbation from the responsive cellular profile.

In the case of a transcriptional state of a cell, even a slight perturbation of a protein activity in a cell is likely to result, through direct or indirect effects, in a measurable change in the transcriptional profile. A reason that disruption in a protein's activity level changes the transcriptional state of a cell is because the previously mentioned feedback systems, or networks, which react in a compensatory manner to infections, genetic modifications, environmental changes, drug administration, and so forth do so in part by altering patterns of gene expression or transcription. As a result of internal compensations, many perturbations to a biological system, although having only a muted effect on the external behavior of the system, can nevertheless profoundly influence the internal response of individual elements, e.g., gene expression, in the cell.

5. Physiological States

A physiological state refers to any measurable state of the subject's physiology. These states include, but are not limited to, nutritional status, biological age and rate of aging, disease, illness, infection, general cardiovascular or pulmonary fitness due to exercise and/or biological age, etc. It is expected that physiological state is somewhat dynamic and that changes in physiological state may be effected by multiple factors including but not limited to diet, exercise, genetic modification, sexual activity, sleep or rest, topical and/or parenteral and/or oral therapies or drugs, infection, or the development of a disease or illness. A physiological state may be deduced or, optionally, defined, in at least some aspects, by the effect of a sample derived from the subject upon the cellular state of a cell contacted with the sample.

Physiological states that are of particular interest are those associated with diet and nutrition, age and rates of aging, disease, and therapies intended to improve the physiological state of animal and human subjects with respect to these states. Dietary and nutritional states refer to the physiological and biological states of a subject that are due to the intake of foods, beverages, and nutritional supplements, such as vitamins, minerals, herbs, etc, or that are associated with human and other molecular profiles that are associated with these states. Biological age and rates of aging refer to physiological and biological states that occur with the passage of time, or that are associated with human and other molecular profiles that are associated with these states. Biological age is highly correlated with chronological age; however, the correlation is not perfect and certain individuals who are “older,” i.e., were born earlier and have a higher chronological age, appear younger and healthier than certain other apparently non-diseased individuals of a younger chronological age. Disease state refers to any abnormal biological state of a subject, or to human and other molecular profiles that are associated with a disease. Any physiological state that is associated with a disease or disorder is considered to be a disease state. As used in the present application, the “level” of a disease or disease state is an arbitrary measure reflecting the progression or state of a disease or disease state. Generally, a disease or disease state will progress through a plurality of levels or stages, wherein the effects of the disease become increasingly severe. The presence or status of these physiological states may be identified by the same collection of biological constituents used to determine any physiological state of the subject. In general but not always, states associated with advanced biological age, aging, and/or disease, will be detrimental to the subject.

A disease state may be a consequence of, for example, a pathogen, including a viral infection (e.g., AIDS, hepatitis B, hepatitis C, influenza, measles, etc.), a bacterial infection, a parasitic infection, a fungal infection, or infection by some other organism. A disease state may also be the consequence of an environmental agent, such as a biological or chemical toxin, or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatoses, breast cancer and other heritable cancers, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York). A disease state may also result from an interaction between genetic predispositions and behaviors of the subject (e.g. diet, exercise, etc.) or environmental influences (e.g. pathogens, carcinogens, etc.). A disease state may also be a set of symptoms of unknown etiology.

Other exemplary diseases include, but are not limited to, diabetes, hypoglycemia, obesity, cancer, hypertension, Alzheimer's disease and other dementias, neurodegenerative diseases, and neuropsychiatric disorders such as bipolar affective disorders or paranoid schizophrenic disorders. In a specific embodiment, the disease, the level or progression of which is determined, or for which therapy is monitored according to the application, is a genetic disease. Thus, in a specific embodiment, the disease is a cancer associated with a genetic mutation, e.g., translocation, deletion, or point mutation (for example, the Philadelphia chromosome).

A therapy or therapeutic regimen, as used herein, refers to a regimen of treatment intended to reduce or eliminate the symptoms associated with less preferable physiological states, such as biological age, aging, toxification, or disease. A therapeutic regimen may comprise, for example, dietary changes, ingestion of dietary supplements, application of topical compounds, genetic therapy, or a prescribed dosage of one or more drugs.

Typically, the effect of a therapy will be beneficial to a biological system in that it will tend to decrease the level of a disease state, or the rate of aging, or toxification of the subject, or tend to increase the physiological fitness of the subject according to objective criteria. However, in many instances, the effect of a therapy will be adverse to a biological system. For example, many therapies, such as drug regimens or chemotherapies, have toxic side effects. In such instances, it is important to monitor adverse effects. Such monitoring may permit adjustment of the therapy, e.g., by reducing dosages or terminating the therapy altogether, to diminish or eliminate one or more of the adverse effects.

Certain physiological states, e.g., biological age, or aging, or disease, will have particular effects on a biological system, and these effects can be measured by using a sample from this biological system to treat cells and analyzing the resulting responsive cellular profile. These physiological states should be recognizable from a signature cellular profile and are referred to herein as “notable physiological states”. The effects, and their resulting profiles, can therefore be correlated to the level of the physiological or disease state. Likewise, drugs or other agents which may be used in a therapy will each have unique effects on the state of a biological system, and on the resulting in vitro molecular profile, which can be correlated to the level of efficacy of a particular therapy.

In an alternative embodiment, the methods of the application may be used to diagnose or screen for the presence of a disease state, or other physiological state.

6. Microarrays

In many embodiments of the application, a nucleic acid microarray may be employed to measure the levels of a plurality of transcripts in a cell or group of cells. Other techniques may also be employed. Some guidelines for the use of microarray technology are set forth below.

Nucleic acid arrays are often divided into microarrays and macroarrays, where microarrays have a much higher density of individual probe species per area. Microarrays may have as many as 1000 or more different probes in a 1 cm area. There is no concrete cut-off to demarcate the difference between micro- and macroarrays, and both types of arrays are contemplated for use with the application. However, because of their small size, microarrays provide great advantages in speed, automation and cost-effectiveness.

Microarrays are known in the art and consist of a surface to which probes that correspond in sequence to gene products (e.g., cDNAs, mRNAs, PCR products, oligonucleotides) are bound at known positions. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

Although in a preferred embodiment the microarray contains binding sites for products of all or almost all genes in the target organism's genome, such comprehensiveness is not necessarily required. Usually the microarray will have binding sites corresponding to at least 100 genes and more preferably, 500, 1000, 4000 or more. In certain embodiments, the most preferred arrays will have about 50-100% of the genes of a particular organism represented. In other embodiments, the application provides customized microarrays that have binding sites corresponding to fewer, specifically selected genes. Microarrays with fewer binding sites are cheaper, smaller and easier to produce. Several exemplary human microarrays are publicly available. The Affymetrix GeneChip HUM 6.8K is an oligonucleotide array composed of 7,070 genes. A microarray with 8,150 human cDNAs was developed and published by Research Genetics (Bittner et al., 2000, Nature 406:443-546).

The probes to be affixed to the arrays are typically polynucleotides. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e. fragments that do not share more than 10 bases of contiguous identical sequence with any other fragment on the microarray). Computer programs are useful in the design of primers with the required specificity and optimal amplification properties. See, e.g., Oligo p1 version 5.0 (National Biosciences). In the case of binding sites corresponding to very long genes, it will sometimes be desirable to amplify segments near the 3′ end of the gene so that when oligo-dT primed cDNA probes are hybridized to the microarray, less-than-full length probes will bind efficiently. Random oligo-dT priming may also be used to obtain cDNAs corresponding to as yet unknown genes, known as ESTs. Certain arrays use many small oligonucleotides corresponding to overlapping portions of genes. Such oligonucleotides may be chemically synthesized by a variety of well known methods. Synthetic sequences are between about 15 and about 500 bases in length, more typically between about 20 and about 70 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, e.g., Egholm et al., 1993, PNA hybridizes to complementary oligonucleotides obeying the Watson-Crick hydrogen-bonding rules, Nature 365:566-568; see also U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al., 1995, Differential gene expression in the murine thymus assayed by quantitative hybridization of arrayed cDNA clones, Genomics 29:207-209). In yet another embodiment, the polynucleotide of the binding sites is RNA.

The nucleic acids or analogues are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995, Science 270:467-470. This method is especially useful for preparing microarrays of cDNA. (See also DeRisi et al., 1996, Nature Genetics 14:457-460; Shalon et al., 1996, Genome Res. 6:639-645; and Schena et al., 1995, Proc. Natl. Acad. Sci. USA 93:10539-11286).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. USA 91:5022-5026; Lockhart et al., 1996, Nature Biotech 14:1675; U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al., 1996, 11: 687-90). When these methods are used, oligonucleotides of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is incorporated in its entirety for all purposes), could be used, although, as will be recognized by those of skill in the art, very small arrays will be preferred because hybridization volumes will be smaller.

The nucleic acids to be contacted with the microarray may be prepared in a variety of ways. Methods for preparing total and poly(A)+ RNA are well known and are described generally in Sambrook et al., supra. Labeled cDNA is prepared from mRNA by oligo dT-primed or random-primed reverse transcription, both of which are well known in the art (see e.g., Klug and Berger, 1987, Methods Enzymol. 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, Nature Biotech. 14:1675). The cDNAs or RNAs can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescent labels are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, 1992, Academic Press San Diego, Calif.).

In another embodiment, a label other than a fluorescent label is used. For example, a radioactive label, or a pair of radioactive labels with distinct emission spectra, can be used (see Zhao et al., 1995, Gene 156:207; Pietu et al., 1996, Genome Res. 6:492). However, use of radioisotopes is a less-preferred embodiment.

Nucleic acid hybridization and wash conditions are chosen so that the population of labeled nucleic acids will specifically hybridize to appropriate, complementary nucleic acids affixed to the matrix. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches).

Optimal hybridization conditions will depend on the length (e.g., oligomer versus polynucleotide greater than 200 bases) and type (e.g., RNA, DNA, PNA) of labeled nucleic acids and immobilized polynucleotide or oligonucleotide. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York, which is incorporated in its entirety for all purposes. Non-specific binding of the labeled nucleic acids to the array can be decreased by treating the array with a large quantity of non-specific DNA—a so-called “blocking” step.

When fluorescently labeled probes are used, the fluorescence emissions at each site of a transcript array can be, preferably, detected by scanning confocal laser microscopy. When two fluorophores are used, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Research 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., 1996, Genome Res. 6:639-645 and in other references cited herein. Alternatively, the fiber-optic bundle described by Ferguson et al., 1996, Nature Biotech. 14:1681-1684, may be used to monitor mRNA abundance levels at a large number of sites simultaneously. Fluorescent microarray scanners are commercially available from Affymetrix, Packard BioChip Technologies, BioRobotics and many other suppliers.

Signals are recorded, quantitated and analyzed using a variety of computer software. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores is preferably calculated. The ratio is independent of the absolute expression level of the cognate gene, but is useful for genes whose expression is significantly modulated by drug administration, gene deletion, or any other tested event.

Transcript arrays reflecting the transcriptional state of a cell of interest may, for example, be generated by hybridizing a mixture of two differently labeled sets of cDNAs to the microarray. One cell is a cell of interest while the other is used as a standardizing control. The relative hybridization of each cell's cDNA to the microarray then reflects the relative expression of each gene in the two cells. For example, to assess gene expression in a variety of breast cancers, Perou et al. (2000, supra) hybridized fluorescently-labeled cDNA from each tumor to a microarray in conjunction with a standard mix of cDNAs obtained from a set of breast cancer cell lines. In this way, gene expression in each tumor sample was compared against the same standard, permitting easy comparisons between tumor samples.

“Delivery” microarrays can be prepared by mechanical microspotting. According to these methods, small quantities of nucleic acids are printed onto solid surfaces. Microspotted arrays prepared by many manufacturers contain as many as 10,000 groups of probes in an area of about 3.6 cm². Other “delivery” approaches include ink-jetting technologies, which utilize piezoelectric and other forms of propulsion to transfer nucleic acids from miniature nozzles to solid surfaces. Inkjet technologies are available through several centers including Incyte Pharmaceuticals (Palo Alto, Calif.) and Protogene (Palo Alto, Calif.). This technology may provide a density of 10,000 spots per cm². See also, Hughes et al. (2001) Nat. Biotechn. 19:342.

Arrays preferably include control and reference probes. Control probes are nucleic acids which serve to indicate that the hybridization was effective. For example, arrays for detection of human transcripts often contain sets of probes for several prokaryotic genes, e.g., bioB, bioC and bioD from biotin synthesis of E. coli and cre from P1 bacteriophage. Hybridization to these arrays is conducted in the presence of a mixture of these genes or portions thereof to confirm that the hybridization was effective. Control nucleic acids included with the target nucleic acids can also be mRNA synthesized from cDNA clones by in vitro transcription. Other control genes that are often included in arrays are polyA controls, such as dap, lys, phe, thr, and trp.

Reference probes allow the normalization of results from one experiment to another, and to compare multiple experiments on a quantitative level. Reference probes are typically chosen to correspond to genes that are expressed at a relatively constant level across different cell types and/or across different culture conditions. Exemplary reference nucleic acids include housekeeping genes of known expression levels, e.g., GAPDH, hexokinase and actin.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases.

Arrays may also contain probes that hybridize to more than one allele or one or more splice variant of a gene. For example the array can contain one probe that recognizes allele 1 and another probe that recognizes allele 2 of a particular gene.

Exemplary techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. No. 5,593,839; U.S. Pat. No. 5,578,832; EP No. 0 728 520; U.S. Pat. No. 5,599,695; EP No. 0 721 016; U.S. Pat. No. 5,556,752; PCT No. WO 95/22058; U.S. Pat. No. 5,631,734; U.S. Pat. No. 6,083,697; and U.S. Pat. No. 6,051,380.

When using commercially available microarrays, adequate hybridization conditions are provided by the manufacturer. When using non-commercial microarrays, adequate hybridization conditions can be determined based on hybridization guidelines that are known in the art, as well as on the hybridization conditions described in the numerous published articles on the use of microarrays. An extensive guide to the hybridization of nucleic acids is found in Tijssen (1993), “Laboratory Techniques in biochemistry and molecular biology-hybridization with nucleic acid probes.”

Following the data gathering operation, the data will typically be reported to a data analysis system. To facilitate data analysis, the data obtained by the reader from the device will typically be analyzed using a digital computer. Typically, the computer will be appropriately programmed for receipt and storage of the data from the device, as well as for analysis and reporting of the data gathered, e.g., subtraction of the background, deconvolution of multi-color images, flagging or removing artifacts, verifying that controls have performed properly, normalizing the signals, interpreting fluorescence data to determine the amount of hybridized target, normalization of background and single base mismatch hybridizations, and the like. Various analysis methods that may be employed in such a data analysis system, or by a separate computer are described herein.

A desirable system for analyzing data is a general and flexible system for the visualization, manipulation, and analysis of gene expression data. Such a system preferably includes a graphical user interface for browsing and navigating through the expression data, allowing a user to selectively view and highlight the genes of interest. The system also preferably includes sort and search functions and is preferably available for general users with PC, Mac or Unix workstations. Also preferably included in the system are clustering algorithms that are qualitatively more efficient than existing ones. The accuracy of such algorithms is preferably hierarchically adjustable so that the level of detail of clustering can be systematically refined as desired.

While the above discussion focuses on the use of arrays for the collection of gene expression data, such data may also be obtained through a variety of other methods, that, in view of this specification, are known to one of skill in the art.

A method for high throughput analysis of gene expression is the serial analysis of gene expression (SAGE) technique, first described in Velculescu et al. (1995) Science 270, 484-487. Among the advantages of SAGE is that it has the potential to provide detection of all genes expressed in a given cell type, whether previously identified as genes or not, provides quantitative information about the relative expression of such genes, permits ready comparison of gene expression of genes in two cells, and yields sequence information that can be used to identify the detected genes. Thus far, SAGE methodology has proved itself to reliably detect expression of regulated and nonregulated genes in a variety of cell types (Velculescu et al. (1997) Cell 88, 243-251; Zhang et al. (1997) Science 276, 1268-1272 and Velculescu et al. (1999) Nat. Genet. 23, 387-388.

For example, gene expression data may be gathered by RT-PCR. mRNA obtained from a sample is reverse transcribed into a first cDNA strand and subjected to PCR. House keeping genes, or other genes whose expression is fairly constant can be used as internal controls and controls across experiments. Following the PCR reaction, the amplified products can be separated by electrophoresis and detected. Taqman™ fluorescent probes, or other detectable probes that become detectable in the presence of amplified product may also be used to quantitate PCR products. By using quantitative PCR, the level of amplified product will correlate with the level of RNA that was present in the sample. The amplified samples can also be separated on a agarose or polyacrylamide gel, transferred onto a filter, and the filter hybridized with a probe specific for the gene of interest. Numerous samples can be analyzed simultaneously by conducting parallel PCR amplification, e.g., by multiplex PCR.

Transcript levels may also be determined by dotblot analysis and related methods (see, e.g., G. A. Beltz et al., in Methods in Enzymology, Vol. 100, Part B, R. Wu, L. Grossmam, K. Moldave, Eds., Academic Press, New York, Chapter 19, pp. 266-308, 1985). In one embodiment, a specified amount of RNA extracted from cells is blotted (i.e., non-covalently bound) onto a filter, and the filter is hybridized with a probe of the gene of interest. Numerous RNA samples can be analyzed simultaneously, since a blot can comprise multiple spots of RNA. Hybridization is detected using a method that depends on the type of label of the probe. In another dotblot method, one or more probes of one or more genes characteristic of disease D are attached to a membrane, and the membrane is incubated with labeled nucleic acids obtained from and optionally derived from RNA of a cell or tissue of a subject. Such a dotblot is essentially an array comprising fewer probes than a microarray.

Another format, the so-called “sandwich” hybridization, involves covalently attaching oligonucleotide probes to a solid support and using them to capture and detect multiple nucleic acid targets (see, e.g., M. Ranki et al., Gene, 21, pp. 77-85, 1983; A. M. Palva, T. M. Ranki, and H. E. Soderlund, in UK Patent Application GB 2156074A, Oct. 2, 1985; T. M. Ranki and H. E. Soderlund in U.S. Pat. No. 4,563,419, Jan. 7, 1986; A. D. B. Malcolm and J. A. Langdale, in PCT WO 86/03782, Jul. 3, 1986; Y. Stabinsky, in U.S. Pat. No. 4,751,177, Jan. 14, 1988; T. H. Adams et al., in PCT WO 90/01564, Feb. 22, 1990; R. B. Wallace et al. 6 Nucleic Acid Res. 11, p. 3543, 1979; and B. J. Connor et al., 80 Proc. Natl. Acad. Sci. USA pp. 278-282, 1983). Multiplex versions of these formats are called “reverse dot blots.”

mRNA levels can also be determined by Northern blots. Specific amounts of RNA are separated by gel electrophoresis and transferred onto a filter which is then hybridized with a probe corresponding to the gene of interest.

The level of expression of one or more genes in a cell may be determined by in situ hybridization. In one embodiment, a tissue sample is obtained from a subject, the tissue sample is sliced, and in situ hybridization is performed according to methods known in the art, to determine the level of expression of the genes of interest. Gene expression may also be monitored by use of a reporter gene (eg. lacZ, cat, GUS, gfp, etc.) linked to the relevant promoter.

A variety of statistical methods are available to assess the degree of relatedness in expression patterns of different genes. Generally, such statistical methods may be broken into two related portions: metrics for determining the relatedness of the expression pattern of one or more gene, and clustering methods, for organizing and classifying expression data based on a suitable metric (Sherlock, 2000, Curr. Opin. Immunol. 12:201-205; Butte et al., 2000, Pacific Symposium on Biocomputing, Hawaii, World Scientific, p.418-29).

In one embodiment, Pearson correlation may be used as a metric. In brief, for a given gene, each data point of gene expression level defines a vector describing the deviation of the gene expression from the overall mean of gene expression level for that gene across all conditions. Each gene's expression pattern can then be viewed as a series of positive and negative vectors. A Pearson correlation coefficient can then be calculated by comparing the vectors of each gene to each other. Pearson correlation coefficients account for the direction of the vectors, but not the magnitudes.

In another embodiment, Euclidean distance measurements may be used as a metric. In these methods, vectors are calculated for each gene in each condition and compared on the basis of the absolute distance in multidimensional space between the points described by the vectors for the gene.

In a further embodiment, the relatedness of gene expression patterns may be determined by entropic calculations (Butte et al. 2000). Entropy is calculated for each gene's expression pattern. The calculated entropy for two genes is then compared to determine the mutual information. Mutual information is calculated by subtracting the entropy of the joint gene expression patterns from the entropy for calculated for each gene individually. The more different two gene expression patterns are, the higher the joint entropy will be and the lower the calculated mutual information. Therefore, high mutual information indicates a non-random relatedness between the two expression patterns.

The different metrics for relatedness may be used in various ways to identify clusters of genes. In one embodiment, comprehensive pairwise comparisons of entropic measurements will identify clusters of genes with particularly high mutual information. A statistical significance for mutual information may be obtained by randomly permuting the expression measurements 30 times and determining the highest mutual information measurement obtained from such random associations. All clusters with a mutual information higher than can be obtained randomly after 30 permutations are statistically significant.

In another embodiment, agglomerative clustering methods may be used to identify gene clusters. In one embodiment, Pearson correlation coefficients or Euclidean metrics are determined for each gene and then used as a basis for forming a dendrogram. In one example, genes were scanned for pairs of genes with the closest correlation coefficient. These genes are then placed on two branches of a dendrogram connected by a node, with the distance between the depth of the branches proportional to the degree of correlation. This process continues, progressively adding branches to the tree. Ultimately a tree is formed in which genes connected by short branches represent clusters, while genes connected by longer branches represent genes that are not clustered together. The points in multidimensional space by Euclidean metrics may also be used to generate dendrograms.

In yet another embodiment, divisive clustering methods may be used. For example, vectors are assigned to each gene's expression pattern, and two random vectors are generated. Each gene is then assigned to one of the two random vectors on the basis of probability of matching that vector. The random vectors are iteratively recalculated to generate two centroids that split the genes into two groups. This split forms the major branch at the bottom of a dendrogram. Each group is then further split in the same manner, ultimately yielding a fully branched dendrogram.

In a further embodiment, self-organizing maps (SOM) may be used to generate clusters. In general, the gene expression patterns are plotted in n-dimensional space, using a metric such as the Euclidean metrics described above. A grid of centroids is then placed onto the n-dimensional space and the centroids are allowed to migrate towards clusters of points, representing clusters of gene expression. Finally the centroids represent a gene expression pattern that is a sort of average of a gene cluster. In certain embodiments, SOM may be used to generate centroids, and the genes clustered at each centroid may be further represented by a dendrogram. An exemplary method is described in Tamayo et al., 1999, PNAS 96:2907-12. Once centroids are formed, correlation are evaluated by, for example, one of the methods described supra.

In operation, the methods and components for receiving gene or protein expression data, the methods and components for analyzing the gene expression data, and the methods and components for presenting information may involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein.

7. Measurement of Other Aspects of Biological State

In various embodiments of the present application, aspects of the biological state other than the transcriptional state, such as the translational state, the activity state, or mixed aspects can be measured in order to obtain therapy and disease state responses. Details of these embodiments are described in this section.

Measurement of the translational state may be performed according to several methods. For example, whole genome monitoring of protein (i.e., the “proteome,” Goffeau et al., supra) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to the action of a disease state or therapeutic effect of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In a preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated by two-dimensional gel electrophoresis systems Two-dimensional gel electrophoresis is well-known in the art and typically involves iso-electric focusing along a first dimension followed by SDS-PAGE electrophoresis along a second dimension. See, e.g., Hames et al, 1990, Gel Electrophoresis of Proteins: A Practical Approach, IRL Press, New York; Shevchenko et al., 1996, Proc. Nat'l Acad. Sci. USA 93:1440-1445; Sagliocco et al., 1996, Yeast 12:1519-1533; Lander, 1996, Science 274:536-539. The resulting electropherograms can be analyzed by numerous techniques, including mass spectrometric techniques, western blotting and immunoblot analysis using polyclonal and monoclonal antibodies, and internal and N-terminal micro-sequencing. Mass spectrometry can be used with other fractionation or separation techniques such as Surface Enhanced Laser Desorption Ionization (SELDI), or Isotope Coded Affinity Tags (ICAT) to achieve a similar result. Using these techniques, it is possible to identify a substantial fraction of all the proteins produced under given physiological conditions, including in cells (e.g., in yeast) exposed to a drug, or in cells modified by, e.g., deletion or over-expression of a specific gene. In the present application either the cellular constituents contained within the cell, or those secreted from the cell into the surrounding milieu, may be measured by one or more of these techniques.

Metabolites or other cellular constituents may also be measured to obtain cellular profiles. Metabolites are measurable using a variety of techniques familiar to those of skill in the art. Mass spectrometry, radioimmunoassay, Nuclear Magnetic Resonance (NMR), and various electrophoretic and chromatography methods can be used to measure the abundances of various and specific metabolites. Radioactively labeled amino acids or other metabolites or nutrients may be added to the assay media, which are taken up by the assay cells and incorporated into cellular constituents. The resulting labeled metabolites can then be quantitated by a variety of means to determine their abundances.

8. Illustration of Certain Methods

Within eukaryotic cells, there are hundreds to thousands of arbitrarily separated signaling pathways that are interconnected. For this reason, perturbations in the function of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “signature” of particular transcript alterations which are related to the disruption of function, i.e., a particular disease state or therapy, even at a stage where changes in activity of the disrupting protein are not directly detectable.

Some of these compensatory changes affect proteins the cell displays on its surface and secretes into the fluids in which it is bathed, e.g., blood or lymphatic fluid. All of the cells of the organism function in a network of interacting pathways that is a systemic analog to the interacting pathways of the individual cells, with respect to the cascades of compensatory changes they elicit in one another. Therefore, it might be expected, for example, that mutations or drugs that alter B cell function, and thereby directly influence the molecular composition of blood, might indirectly be detected in urine or saliva. In certain physiological states, such as those associated with various biological ages or diseases, as two examples, these alterations in the composition of biological samples can elicit molecular profiles from cells that may be correlated to the physiological state of the subject with respect to the parameter of interest, such as in the given examples, the biological age or disease state.

In certain embodiments, the “analogous subject” from whom perturbation profiles are obtained may be the same individual (i.e., the same organism or patient) as the subject upon whom a physiological state or the effect of a therapy is being monitored. For example, intensity correlation profiles may be obtained from an individual at two biological ages, or during episodes of disease, remission, or recurrence.

In other embodiments, it is desirable to monitor the effect of a plurality of therapies upon a subject, for example a regimen comprising drugs A, B, and C. In such embodiments, intensity correlation profiles could be obtained first for drug A, by monitoring the effect of drug A, alone, on the same subject and correlating that effect with measurements of cellular constituents from an assay cell exposed to a biological sample from that subject. Likewise, intensity correlation profiles could next be obtained in the same manner for drug B alone, and for drug C alone. The intensity correlation profiles could then be used to monitor the cumulative effect of the combination of therapies (in this example the combination of drugs A, B, and C) upon that same subject.

In still other embodiments, intensity correlation profiles are obtained for one or more physiological states and/or for one or more therapies and are calibrated to a clinical effect or effects. Exemplary clinical effects include, but are not limited to, blood pressure, body temperature, levels of blood or urine glucose or other metabolites, hormonal levels (including e.g., testosterone, estrogen, insulin, leptin, IGF-1, DHEA, etc.), cholesterol levels (including, e.g., HDL and LDL levels), viral load levels, blood hematocrit levels, white cell count, tumor size etc. In fact, any measurement of a patient's biochemical and/or physiological state that may be readily obtained in a clinical setting is a measurement of a clinical effect.

In such embodiments, the levels of one or more physiological states can be determined and/or monitored in a patient by monitoring the patient's inferential molecular profile and comparing it to the clinical effect or effects that are calibrated to intensity correlation profiles for the one or more physiological states. Likewise, one or more drug therapies may be monitored in a patient by monitoring the inferential molecular profile of a patient undergoing the therapy (or therapies) and comparing it to the clinical effect or effects that are calibrated to intensity correlation profiles for the one or more therapies. A desirable clinical effect can then be readily achieved for the patient by adjusting the therapy (or therapies) until the patient's inferential molecular profile matches the profile obtained for the desired clinical effect.

Although, much of the description of this application is directed to measurement and modeling of gene expression data in an assay cell, this application is equally applicable to measurements of other aspects of the cellular constituents of assay cells, such as protein abundances, modifications, or activities, DNA modifications, protein-protein interactions, or protein-DNA interactions. Methods for direct measurement of protein modification and activity are well known to those of skill in the art. Such methods include, e.g., methods that depend on having an antibody ligand for the protein, such as Western blotting (see, e.g., Burnette, 1981, A. Anal. Biochem. 112:195-203). Such methods also include enzymatic activity assays, which are available for most well-studied protein drug targets, including, but not limited to, HMG CoA reductase (Thorsness et al., 1989, Mol. Cell. Biol. 9:5702-5712), and calcineurin (Cyert et al., 1992, Mol. Cell. Biol. 12:3460-3469). An example of turning off a specific gene function by turning off the controllable promoter, and correlating this with protein depletion via Western blotting is given in Deshaies et al., 1988, Nature 332:800-805.

Methods for the analysis of DNA modifications are well known to those skilled in the art, as are methods for measuring protein-protein or protein-DNA interactions. (As examples, see Fields S, Song O., Nature Jul. 20, 1989;340(6230):245-6; Tavazoie S, Church G M., Nat Biotechnol 1998 June;16(6):566-71; Ren B, et al., Science Dec. 22, 2000;290(5500):2306-9.)

### EXAMPLES

Exemplary embodiments of the application may comprise one or more of the following phases: obtaining biological samples from a subject, in vitro treatment of cells with samples, preparation of cDNA and hybridization to arrays and data analysis. Each phase may comprise one or more of the exemplary protocol steps provided below.

Obtaining biological samples from a subject:
1. The evening before the assay the subject may eat a meal of standard composition and size (weight, volume, or calories) within an hour of some set time before the assay.
2. For hydration of the subject, the morning of the assay the subject may drink a glass of water of fixed volume at a fixed length of time before the sample is obtained.
3. At a set time a biological sample is obtained from the subject. Other times may be used but circadian variation occurs in samples and this effect can be minimized by sampling at an invariant time. The sample may be blood, saliva, urine, and/or fluid scraped or drawn from the skin, although it may be of other types. Fluid may be obtained from the skin by disruption of the barrier function by skin tape stripping or dermabrasion. In the following exemplary steps either blood serum or plasma is used as the sample and is obtained by the use of standard phlebotomy techniques. The use of serum requires the use of a blood collection tube that does not contain agents that interfere in clotting, such as sodium citrate, while plasma is often separated from blood cells by the use of vials containing such agents. The collection tube is preferably of the vacuum type. The blood may be processed in a number of ways, for example, serum may be obtained from the blood by “off the clot” methods known to those skilled in the art. Plasma may be separated from blood cells, and is an appropriate biological sample for use in the in vitro treatment of cells for generating molecular profiles, as provided for by the primary methods of the application, and the separated blood cells can be used to generate separate molecular profiles, as provided by the secondary methods of the application. A number of samples of blood may be obtained but the total volume of blood taken from the subject in one session preferably should not exceed 0.15 ml per pound of body weight. At least five days recovery should be allowed if the maximum volume is taken. The following volumes are useful for these different samples: blood, 5 to 10 ml; saliva, 0.5 to 2 ml; urine, 1 to 10 ml; skin scraping or secretion, 10 to 500 microliters. This sample is referred to as the t0 sample (time=0). Immediately upon taking the sample a timer is started for timing the taking of subsequent samples.
4. The t0 sample is either set aside under defined conditions for use in the performance of the assay at some later time, or it is extracted, fractionated, or taken whole, and frozen in liquid nitrogen for later use as described below.
5. After (preferably immediately after) the t0 sample is taken from the subject the subject should be treated in some defined manner. For example, this may be treatment with a drug, beverage, food, or food supplement of fixed dosage, or the subject might undertake some defined activity in order to alter the physiological state, such as exercise, sleep, or sexual arousal or activity, etc.
6. After a fixed amount of time from t0, generally 60 to 180 minutes, a second sample is taken, and the sample is denoted by “t” followed by the number of minutes from t0 the sampling was begun. For example, if the sample is taken 90 minutes after the t0 sample then this second sample is denoted to be the t90 sample. Occasionally this second sample will be taken without treatment of the subject. This is a reference or calibration sample that indicates the change in the physiological state of the subject in the absence of treatment.
7. The samples taken from the subject are treated in identical ways. For example, if blood is drawn for the purpose of obtaining serum, the blood should be allowed to clot in vitro, and the serum and other blood should be separated by centrifugation. Once this is achieved the serum should be poured off and immediately frozen. This same procedure should be followed for sample 2 with each step taking the same amount of time. Only the time spent in the frozen state differs between samples. The sample may be sterile filtered prior to freezing or prior to use.

In vitro treatment of cells with the samples
1. A population of standard assay cells is grown in culture using techniques familiar to those skilled in the art (HeLa, HEK293, TERT-immortalized fibroblasts, for example). Several different variations of culture condition may be used: for example, serum vs. serum free, attached vs. suspension, etc. The particular details of culturing depend on cell type, and appropriate culturing conditions are known in the art. In general, cells grown under serum-free or low serum conditions in suspension are preferred, as this provides the most sensitivity and ease of handling upon addition of sample. A single culture of cells is grown for aliquoting, or multiple cultures can be grown and pooled for this purpose. As an example, suspension grown 293 cells are considered in the remainder of these exemplary protocol steps.
2. Cells grown may be aliquoted to individual dishes, or to individual wells of multi-well dishes, and grown for not more than 48 hours to ensure dish-to-dish or well-to-well homogeneity. The number of cells aliquoted into each dish should be similar and the range of cell density should be in the range of 5×10\hat{ }4 to 3×10\hat{ }5 cells per ml of media. Typical culture conditions are 37° C. and 5% to 10% CO₂, but may vary depending on specific cell type.
3. Individual samples are added to individual wells (typically<1 mL-5 mL for serum, or in the range of 1% to 100% of the final media concentration), or the samples are mixed with other media components and/or samples. Cells are cultured for a predetermined length of time in the presence of sample (usually 2-8 hr), or the sample can be used in a “pulse/chase” manner in which the sample is added to the media, and subsequent to the addition the media is replaced with fresh media absent the sample.
4. Following incubation subsequent to treatment, cells are spun down by centrifugation at 4° C., 800×g for 5 min.
5. Media will be removed from the cell pellet by aspiration and cells are lysed by addition of lysis buffer.
6. Following lysis, purified RNA is obtained by immediate purification by any one of a variety of methods, e.g., by the use of a Qiagen RNeasy column. Total RNA may be stored at −20° C. Total RNA may be further fractionated to yield mRNA which is useful in certain applications.

Obtaining cells from the subject
1. Cells taken from the subject for generating molecular profiles should be handled in some specified fashion. For example, blood cells used for the diagnosing the presence a pre-disease state, e.g., insulin resistance, should be handled in a manner as similar as possible to the handling of cells used to generate training sets and inferential molecular profiles.
2. Fat cells may be obtained from the patient by means of liposuction. Skin cells or other endothelial or epithelial cells may be obtained by punch biopsy or other biopsy techniques. Blood cells may be obtained from the patient by standard phlebotomy techniques. Blood cells may be fractionated, clotted, or separated by techniques familiar to those skilled in the art.
3. RNA can be extracted from all tissue types using techniques familiar to those skilled in the art, and may be further purified or fractionated.

Preparation of cDNA and hybridization to arrays
1. RNA obtained from treated cells is reverse transcribed into cDNA using an oligo-dT primer and labeled with a fluorescent dye (cy3 for example).
2. Labeled cDNA produced from RNA obtained from cells treated with a particular sample (t90 for example) is hybridized to a microarray containing oligonucleotides or PCR products representing many different human genes (or genes from the same organism the cells are derived from). cDNA produced from RNA obtained from cells treated with another sample, e.g. t0, and labeled differently, e.g., with a different fluorescent dye (cy5 for example), is hybridized concurrently to the same array.
3. Fluorescence levels for each dye at each oligonucleotide position on the array are measured, normalized, and used to determine relative abundance of a particular mRNA transcript in treated vs. untreated cells. In this way, a “transcript profile” is obtained for each sample relative to another sample, for example t90/t0.

Data analysis
1. Transcriptional profiles from cells treated with different samples are compared to determine which mRNA transcripts change abundance in response to a particular therapeutic regimen. Genes that are observed to repeatedly demonstrate altered transcription in response to a particular physiological state or therapeutic regimen define a set of biomarkers make up an “inferential set of cellular constituents” or “inferential set”. Gene transcripts of an inferential set may be examined in other responsive cellular profiles to predict the physiological state of a subject from which a sample is obtained.
2. Inferential sets will be generated for many different treatments including dietary alterations (e.g. calorie restriction), dietary supplementation, behavioral and lifestyle modification (alcohol consumption, smoking, etc.), physical stress, etc. Likewise, inferential sets may also be obtained from individuals with differing genetic backgrounds, where the inferential set provides the identity of cellular constituents that are informative of genetic background. This type of inferential set may be referred to as a “genetic inferential set”.
3. The various inferential sets will be combined into an “inferential set database”. Transcriptional profiles (and other responsive cellular profiles) will be combined into a “profile database”.
4. Statistical and bioinformatic techniques will be used to compare database profiles and inferential sets to patient profiles for diagnostic and therapeutic purposes. These techniques are familiar to those skilled in the art and multiple useful approaches to statistical and data analysis are found in the relevant literature (see, e.g., Eisen M B, et al., Proc Natl Acad Sci U S A Dec. 8, 1998;95(25):14863-8; Hughes, T, et al., Cell Jul. 7, 2000;102(1):109-26; Friend and Stoughton, U.S. Pat. No. 6,218,122).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Also incorporated by reference are the following U.S. Pat. Nos. 3,091,216; 5,510,270; 5,556,752; 5,569,588; 5,578,832; 5,633,161; 5,663,071; 5,674,739; 5,677,125; 5,695,937; 5,702,902; 5,707,807; 5,721,337; 5,721,351; 5,723,290; 5,741,666; 5,746,204; 5,759,776; 5,935,060; 5,965,352; 6,084,742; 6,090,004; 6,132,969; 6,146,830; 6,210,970; 6,210,902; 6,222,093; 6,329,209; 6,218,122; 6,370,478; 6,324,479; 6,372,431; the following Foreign Patent Documents: 0 534 858 A1; and the following publications: Blanchard et al., 1996, “High-density oligonucleotide arrays”, Biosensors & Bioelectronics 11:687-690; Blanchard and Hood, 1996, “Sequence to array: probing the genome's secrets”, Nature Biotechnol. 14:1649; deRisi et al., 1996, “Use of a cDNA microarray to analyse gene expression patterns in human cancer”, Nature Genet. 14:457-460; Lockhart et al., 1996, Expression monitoring by hybridization to high-density oligonucleotide arrays, Nature Biotechnology 14:1675-1680; Heller, R A et al, “Discovery and analysis of inflammatory disease-related genes using cDNA microarrays”, Proc. Natl. Acad. Sci. USA, Mar. 18, 1997, vol. 94, No. 6, pp. 2150-2155; Schena et al., 1995, “Quantitative monitoring of gene expression patterns with a complementary DNA microarray”, Science 270:467-470; Schena et al., 1996, “Parallel human genome analysis: microarray-based expression monitoring of 1000 genes”, Proc. Natl. Acad. Sci. USA 93:10614-10619; Shalon et al., 1996, “A DNA microarray system for analyzing complex DNA samples using two-color fluorescent probe hybridization”, Genome Res. 6:639-645; Shevchenko et al., 1996, “Linking genome and proteome by mass spectrometry: large-scale identification of yeast proteins from two dimensional gels”, Proc. Natl. Acad. Sci. USA 93:14440-14445; Velculescu et al., 1995, “Serial analysis of gene expression”, Science 270:484-487; Yatscoff et al., 1996, “Pharmacodynamic monitoring of immunosuppressive drugs”, Transpl. Proc. 28:3013-3015; Zhao et al., 1995, “High-density cDNA filter analysis: a novel approach for large-scale, quantitative analysis of gene expression,” Gene 156:207-213.

### EQUIVALENTS

While specific embodiments of the subject applications have been discussed, the above specification is illustrative and not restrictive. Many variations of the applications will become apparent to those skilled in the art upon review of this specification and the claims below. The full scope of the applications should be determined by reference to the claims, along with their full scope of equivalents, and the specification, along with such variations.

1. A method of generating and optionally using a responsive cellular profile corresponding to a biological state, the method comprising:
a) providing a first sample from a first subject, wherein the first subject has a first biological state;
b) providing a first cell population;
c) contacting the first sample with the first cell population in vitro;
d) detecting a plurality of cellular constituents of the first cell population to generate a first responsive cellular profile, wherein the first responsive cellular profile corresponds to the first biological state.
e) providing information that describes the first biological state;
f) optionally, creating a database linking the information that describes the first biological state to the first responsive cellular profile.

2. The method of claim 1, further comprising:
g) providing n additional samples from one or more subjects that may include the first subject, wherein each of the first subject and/or one or more additional subjects has one or more additional biological states;
h) providing n additional cell populations;
i) contacting each of the n additional samples with one of the n cell populations in vitro;
j) detecting a plurality of cellular constituents of each of the n cell populations to generate n additional responsive cellular profiles, wherein each of the n additional responsive cellular profiles corresponds to an additional biological state.

3. The method of claim 2, further comprising:
a) comparing the first responsive cellular profile to a second responsive cellular profile, thereby generating a similarity index.
b) optionally, comparing two or more responsive cellular profiles and their respective corresponding biological states, thereby generating an inferential set.
c) optionally, providing information that describes the biological state corresponding to one or more of the responsive cellular profiles; and creating a database linking the information that describes each biological state to the corresponding responsive cellular profile.

4. The method of any of claims 1-3, wherein each responsive cellular profile is of the same type, and wherein the type is selected from the group consisting of: transcriptional profile, translational profile, protein activity profile and mixed profile.

5. The method of any of claims 1-3 wherein each cell population comprises a cell type selected from the group consisting of: fibroblast, blood, fat, endothelial, epithelial, lymph or lymphatic system, skin, liver, muscle, brain, bone, neuronal, kidney, breast, lung, hematopoeitic stem cells and other stem cells, undifferentiated or partially differentiated cell types, any cell type from eukaryotes, prokaryotes, or archae.

6. The method of any of claims 1-3 wherein each biological sample is selected from the group consisting of: blood, urine, mucous, tears, saliva, sweat, feces, peritoneal fluid, cerebrospinal fluid, sebum, breast milk, amniotic fluid, lymph, blister fluid, pus, pleural fluid, semen, synovial fluid, a different bodily fluid, tissue secretions, tissue extracts, tissue homogenate, cellular secretions or extracts, an extract of any of the preceding, and a fractionate of any of the preceding.

7. The method of any of claims 1-3 wherein each cell population comprises a panel of multiple, separately obtained cell types, and wherein the cellular constituents of the multiple, separately obtained cell types are measured together.

8. The method of any of claims 1-3 wherein each cell population comprises a panel of multiple, separately obtained cell types, and wherein the cellular constituents of the multiple, separately obtained cell types are measured separately.

9. The method of any of claims 1-3 wherein the biological state is a state of dietary or nutritional health.

10. The method of claim 9, wherein the state of dietary or nutritional health is selected from the group consisting of: vitamin sufficiency/deficiency, mineral sufficiency/deficiency, other metabolite balance, imbalance, or flux, calorie or caloric restriction, caloric maintenance, caloric overabundance, metabolism, anabolism, catabolism, fuel or energy balance, imbalance, or flux, vegetarianism, a high protein diet, a high fat diet, a high carbohydrate diet, varying ratios of protein, carbohydrates, fat and fiber, varying types of dietary fiber, different amounts and types of protein, fat, carbohydrate, or fiber, the administration of different fuel or energy sources by various means including orally, intravenously, parenterally, enterally, subcutaneously, or topically, varying blood glucose levels, blood glucose kinetics, insulin levels, insulin kinetics, and growth factor levels and kinetics.

11. The method of any of claims 1-3 wherein the biological state is a non-disease state.

12. The method of claim 11 wherein the non-disease state is selected from the group consisting of: chronological or biological age, rate of aging, healthiness or healthfulness, ion or electrolyte balance, imbalance, or flux, presence or absence and proportion of senescent cells in cells, organs and tissues, presence or absence and proportion of damaged, dying, dead, or apoptotic cells in cells, organs and tissues, hormonal balance, flux or imbalance, or flux, pregnancy, menopause, fatigue, chronic fatigue or unexplained lethargy, pain, headache or migraine headache, swelling or edema, weakness, dizziness or disorientation, loss of balance, colonization or infection by a microbial pathogen, or blindness or other disability.

13. The method of any of claims 1-3, wherein the biological state is selected from the group consisting of: a pre-disease state and an early disease state.

14. The method of claim 13, wherein the biological state is selected from the group consisting of: pre-diabetes, insulin resistance, glucose intolerance, obstructive lung and other pulmonary difficulties pre-cancer, pre-metastatic cancer, pre-cancer from tobacco use, pre-emphysema, pre-stroke, cardiovascular disease, pre-heart disease, pre-coronary heart disease, pre-liver or kidney disease, dementia, pre-Alzheimer, or apparently disease-free but aged.

15. The method of any of claims 1-3 wherein the biological state is selected from the group consisting of: allergic reaction, sensitivity to a chemical or biological agent, and or toxification or intoxication with a toxic agent or a drug.

16. The method of any of claims 1-3 wherein a therapy, treatment, intervention, or perturbation is used or prescribed to attempt to change the biological state to another biological state as measured by the invention described herein.

17. The method of claim 16, wherein a therapy, treatment, intervention, or perturbation is selected from the group consisting of: dietary change, the administration or application of fuel sources to the patient or subject, exercise, the administration of herbs or dietary supplements, drugs, the administration of natural or synthetic products, lifestyle change, surgery, exercise, physical therapy, acupuncture, chiropractic, addition or subtraction from the subject of biological agents such as living cells, proteins or other metabolites, or genetic constructs.

18. A method of generating and optionally using a responsive cellular profile corresponding to a biological state comprising:
a) providing a first tissue or cellular sample from a first subject, wherein the first subject has a first biological state;
b) detecting a plurality of cellular constituents of cells of the first cellular sample to generate a first responsive cellular profile, wherein the first responsive cellular profile corresponds to the first biological state.

19. The method of claim 18, further comprising:
c) providing n additional tissue or cellular samples from one or more subjects that may include the first subject, wherein each of the first subject and/or one or more additional subjects has one or more additional biological states;
d) detecting a plurality of cellular constituents of each of the n tissue or cellular samples to generate n additional responsive cellular profiles, wherein each of the n additional responsive cellular profiles corresponds to an additional biological state.

20. The method of claim 19, further comprising:
a) comparing the first responsive cellular profile to a second responsive cellular profile, thereby generating a similarity index.
b) optionally, comparing two or more responsive cellular profiles and their respective corresponding biological states, thereby generating an inferential set.
c) optionally, providing information that describes the biological state corresponding to one or more of the responsive cellular profiles; and creating a database linking the information that describes each biological state to the corresponding responsive cellular profile.

21. A method of determining the presence, intensity, stage, or level, of a biological state of a subject, the method comprising:
a) providing a first responsive cellular profile corresponding to the physiological state of the subject;
b) providing one or more additional responsive cellular profiles corresponding to one or more known related physiological states of the same or different subjects;
c) comparing the first responsive cellular profile to the one or more additional responsive profiles to identify similar cellular profiles, wherein the physiological state of the subject is similar to the known physiological state that corresponds to the similar cellular profiles.

22. A method of determining the presence, intensity, stage, or level, of a biological state of a subject, the method comprising:
a) providing a plurality of responsive cellular profiles corresponding to a plurality of known related biological states of one or more subjects;
b) analyzing the plurality of responsive cellular profiles corresponding to a plurality of known related biological states to generate an inferential set;
c) providing a responsive cellular profile corresponding to a subject having an unknown physiological state;
d) using the inferential set to predict the biological state of the subject having an unknown biological state.

23. The method of claim 21 or 22, wherein the related biological states are varying degrees of severity of a disease state.

24. The method of claim 21 or 22, wherein the related biological states are varying levels of positive or negative effects of a therapeutic regimen.

25. The method of claim 21 or 22, wherein comparing the first responsive cellular profile to the one or more additional responsive profiles comprises determining a measure of correlation between the profiles that are compared.

26. The method of claim 22, wherein the inferential set is generated using a statistical method selected from the group consisting of: a correlative method and a clustering method.

27. The method of claim 22, wherein the inferential set comprises a regression curve and where using the inferential set comprises interpolating or extrapolating to calculate a predictive profile most similar to the responsive cellular profile corresponding to a subject having an unknown physiological state.

28. A method of determining the presence, intensity, stage, or level, of one or more physiological states of a subject, said method comprising:
a) providing an inferential set comprising, for each physiological state, a set of levels of a plurality of cellular constituents, wherein the variation in the levels of the cellular constituents is predictive of physiological state;
b) providing a responsive cellular profile for the subject;
c) extracting from the inferential set one or a combination of calculated predictive profiles for which similarity is greatest between the responsive cellular profile and the calculated predictive profiles, wherein each calculated predictive profile corresponds to a predicted level of a physiological state of the subject.

29. The method of claim 28 wherein each predicted level of a physiological state of the subject is a level which minimizes the value of an objective function of the difference between the responsive cellular profile and a calculated predictive profile extracted from the inferential set.

30. A method of determining a level of effect of one or more therapies upon a subject, said method comprising:
a) providing an inferential set comprising, for each therapy, a set of levels of a plurality of cellular constituents, wherein the variation in the levels of the cellular constituents is predictive of the level of effect of a therapy;
b) providing a responsive cellular profile for the subject;
c) extracting from the inferential set one or a combination of calculated predictive profiles for which similarity is greatest between the responsive cellular profile and the calculated predictive profiles, wherein each calculated predictive profile corresponds to a predicted level of effect of a therapy on the subject.

31. The method of claim 30 wherein the level of effect of a single therapy is determined.

32. The method of claim 30 wherein the inferential set is correlated to levels of effect of each of the therapies by calibrating the set of levels of a plurality of cellular constituents to one or more defined effects.

33. The method of claim 32 wherein the therapies are adjusted until the responsive cellular profile matches a calculated predictive profile derived from the calibrated inferential set at a desired level of the one or more defined effects.

34. The method of claim 30 wherein one or more of the therapies comprises administration of a pharmacologically or biologically active agent to the subject such as a drug, an antioxidant, a prehormone, a hormone or mixture of hormones, exogenous cell or cells, exogenous tissues, nucleic acids, proteins, or other metabolites, to the subject.

35. The method of claim 30 wherein one or more of the therapies comprise administration of a beverage, a food, a fuel or energy source, or one or more food ingredients to the subject.

36. The method of claim 35, wherein the one or more food ingredients comprises a substance selected from the group consisting of: cellular constituents, fats, proteins, sugars, complex carbohydrates, vitamins, minerals, herbs, herbal supplements, food colorings, food additives and food spices.

37. The method of claim 30 wherein said subject has a disease state, and the effect of at least one of the one or more therapies reduces or eliminates the symptoms of the disease state in the subject.

38. The method of claim 30 wherein the effect of at least one of the therapies is an adverse effect.

39. The method of claim 38 wherein the adverse effect is an allergic or toxic effect.

40. The method of claim 30 wherein the predicted level of effect of a therapy is a level which minimizes the value of an objective function of the difference between the responsive cellular profile and a calculated predictive profile extracted from the inferential set for each predicted level of effect of the therapy.

41. The method of claim 28 or 30 wherein the subject is a mammal.

42. The method of claim 28 or 30 wherein the plurality of cellular constituents comprises abundances of a plurality of RNA species present in said cells or cell types, and wherein the responsive cellular profile comprises a transcriptional profile.

43. The method of claim 42 wherein the abundances are measured by a method comprising contacting a microarray with RNA from the cells, or with cDNA derived therefrom.

44. The method of claim 42 wherein the transcriptional profile is generated by a method comprising contacting one or more gene transcript arrays (i) with RNA, or with cDNA derived therefrom, from said cell or cell type treated with biological samples obtained from said subject and (ii) with RNA or with cDNA derived therefrom, from a second cell or cell type treated with biological samples obtained from either (a) said subject from a different time or treated with a different intervention, treatment or therapy, or (b) with RNA or with cDNA derived therefrom, from a second cell or cell type treated with biological samples obtained from a second subject with or without the same intervention, treatment or therapy.

45. The method of claim 28 wherein the set of levels of a plurality of cellular constituents comprises RNA species known to be increased or decreased in a cell in response to perturbations correlated to the physiological state.

46. The method of claim 30 wherein the set of levels of a plurality of cellular constituents comprises RNA species known to be increased or decreased in a cell in response to perturbations correlated to the level of effect of the therapy.

47. The method of any of claims 1-3 wherein the biological sample comprises of combined samples, or a pool of samples, from one or more individual subjects.

48. The method of any of claims 1-3 or 18-20, wherein the information concerning the physiological state of the subject results from a comparison of their responsive cellular profile to another responsive cellular profile obtained by treatment of an cell population with one or more physical or bioactive agents.

49. The method of claim 48, wherein the one or more physical or bioactive agents are selected from the group consisting of: heat, radiation, drugs, hormones, vitamins, proteins and peptides.

50. The method of any of claims 1-3 or 18-20, further comprising:
a) providing a calibration cell population or cellular sample;
b) contacting the calibration cell population or cellular sample with a calibration agent;
c) detecting a plurality of cellular constituents of the calibration cell population to generate a calibration responsive cellular profile, wherein the calibration responsive cellular profile corresponds to a biological response associated with the calibration agent.

51. The method of claim 50, wherein the calibration agent is a bioactive agent has a predictable effect on a biological pathway or genetic network of interest.

52. The method of claim 50, wherein the calibration agent is selected from the group consisting of: an siRNA, a hormone, and a drug.

53. The method of claim 50, further comprising comparing one or more responsive cellular profiles with the calibration responsive cellular profile.

54. The method of claim 50, wherein the calibration agent is a cell or cell secretion derived from a cell type selected from the group consisting of: cells from tumors of breast cancer, lung cancer, prostate, colorectal, lymphoma, infected cells, toxified cells.
